(19) United States
(12) Patent Application Publication
(10) Pub. No.: US 2013/0101642 A1
(43) Pub. Date: Apr. 25, 2013

(54) METHODS AND COMPOSITIONS FOR TREATING ORAL MUCOSITIS

(71) Applicant: Texas Enterosorbents Inc., Bastrop, TX (US)

(72) Inventors: Robert H. Carpenter, Mansfield, TX (US); Richard M. Scruggs, Houston, TX (US)

(73) Assignee: Texas Enterosorbents Inc., Bastrop, TX (US)

(21) Appl. No.: 13/656,322

(22) Filed: Oct. 19, 2012

Related U.S. Application Data
(60) Provisional application No. 61/549,585, filed on Oct. 20, 2011.

(51) Int. Cl. A61K 33/06; A61P 11/04; A61P 29/00; A61K 9/14; A61K 9/10

(52) U.S. Cl. 424/400; 424/684

(57) ABSTRACT

The present disclosure relates generally to the field of oral mucositis. More particularly, methods and compositions for treating, ameliorating and/or preventing oral mucositis are provided. Methods and compositions for treating, ameliorating and/or preventing oral mucositis are also provided.

### CROSS-REFERENCE TO RELATED APPLICATIONS

The present application claims the benefit of priority to U.S. Provisional Patent Application No. 61/549,585, filed Oct. 20, 2011, which is incorporated herein by reference in its entirety.

### TECHNICAL FIELD

The present disclosure relates to methods and compositions for preventing, treating, ameliorating and/or reducing the severity of oral mucositis. More particularly, the disclosure relates to methods for preventing, treating, ameliorating and/or reducing the severity of oral mucositis by providing a composition comprising a non-swelling or low-swelling calcium aluminosilicate clay with an average particle size of less than about 100 microns, referred to herein as CASAD or CASAD UPSN.

### BACKGROUND

Oral mucositis is a condition characterized by swelling, irritation, and discomfort of mucosal linings such as those of the alimentary and gastrointestinal tract, including oral and oralpharyngeal cavities, as well as nasal, optical, vaginal and rectal mucosa. Oral mucositis can result in mouth and throat sores, diarrhea, abdominal cramping and tenderness, and rectal ulcerations. As an inflammation of mucous membranes which often involves infection and/or ulceration, oral mucositis is a serious and often very painful disorder.

Oral mucositis often develops as a complication of chemo- or radiation therapy for cancer. The goal of radiation and chemotherapy in cancer treatment is to kill rapidly dividing cancer cells; unfortunately, other rapidly dividing cells are killed by the treatment as well, including epithelial cells of the mucous membranes lining regions such as the gastrointestinal tract, leading to oral mucositis. Exposure to radiation and/or chemotherapeutics often results in significant disruption of cellular integrity in mucosal epithelium and the underlying connective tissue, leading to inflammation, infection and/or ulceration at mucosal sites such as, for example, in the mouth, throat and other portions of the GI tract.

Oral mucositis adversely impacts the quality of life of cancer patients in several ways. Patients may experience intense pain, nausea and gastro-enteritis. The mouth and throat sores of oral mucositis can cause significant pain and make it difficult to eat, drink, and even take oral medication. In general, symptoms of oral mucositis appear within five to ten days after the start of cancer treatment and can last several weeks after cessation of treatment. The incidence of oral mucositis, as well as its severity, depends on factors such as the type and duration of the cancer treatment. Oral mucositis occurs, for example, in virtually all patients who are treated by irradiation of the head and neck. It is also highly prevalent in patients treated with high dose chemotherapy and/or irradiation for the purpose of myeloablation, in preparation for stem cell or bone marrow transplantation. The severity of oral mucositis can limit subsequent doses of chemotherapy or radiation. Efforts to counter the discomforts of oral mucositis can lead to disruptions in cancer treatment, alterations in treatment dosages, or shifting to different modes of treatment. Severe oral mucositis can also lead to the need for parenteral nutrition or hospitalization for several weeks (or more) of intravenous feeding as a result of the mouth ulcers, cramps, extreme pain, gut denuding and severe diarrhea. Thus, the development of effective approaches to preventing and treating oral mucositis is therefore important for improving the care of cancer patients.

Oral mucositis (OM) is an acute, painful, costly and sometimes debilitating complication of some cancer therapies. Oral mucositis is prevalent in patient populations with head and neck malignancies being treated with radiation therapy. The oral cavity is lined with mucosal epithelium, and exposure to radiation and/or chemotherapeutics results in the disruption of cellular integrity leading to the development of ulcerative lesions. The oral mucositis can be mild requiring little intervention, to severe (hypovolemia, electrolyte abnormalities, and malnutrition) that may result in fatal complications—and this condition affects a significant fraction of cancer patients world-wide.

Oral mucositis usually occurs after the second week of radiation therapy, with severe symptoms usually resolving within six weeks following completion of therapy. In severe cases, oral mucositis can be extremely painful, preventing the patient from eating, and requiring hospitalization for hydration, narcotics for pain, and/or total parenteral nutrition. Pain resulting from oral mucositis is so severe that it is often cited by cancer patients as the primary reason for discontinuing treatment. Patients suffering from oral mucositis have reported feeling as if they were drinking scalding hot water and scraping the inside of their mouth with coarse sand paper followed by running their tongue over a cheese grater. Oral mucositis can also be life-threatening because oral ulcerations can permit the entry of bacteria into the bloodstream, which can lead to fatality in the case of sepsis in a patient already immune-compromised by treatment for cancer. Oral mucositis is therefore a significant risk factor for life-threatening systemic infection; the risk of systemic infection is exacerbated by concomitant neutropenia, which is another complication associated with chemotherapy. Patients with oral mucositis and neutropenia have a relative risk for a life-threatening systemic infection that is at least four times greater than that of individuals without oral mucositis.

The onset of oral mucositis usually involves a four-phase process: the primary phase is inflammatory/vascular in nature, marked by cytokine release from the epithelium brought on by damage caused by radiation and/or chemotherapy. The second phase, referred to as the epithelial phase, is signaled by atrophy and ulceration of the mucosal epithelium. The third phase is defined as the ulcerative/bacterial phase represented by ulcerative lesions that are prone to bacterial infection further compromising the patients' immune system. These painful lesions often limit a patient's ability to eat and drink and in some cases require hospitalization. The presence of these lesions can also interrupt scheduled chemotherapy and/or radiation treatments. The last phase, the healing phase, is characterized by a proliferation and differentiation of epithelium as well as bacterial control.

Virtually all patients treated for tumors of the head and neck, patients receiving radiation along the GI tract, and around 40% of those subjected to radiation therapy and/or chemotherapy for tumors in other locations (leukemias or lymphomas) develop mucositis affecting the oral cavity (Minerva Stomatol. 2002: 51:173-86). Oral mucositis also affects 76-100 percent of patients receiving higher doses of chemotherapy for bone marrow transplantation. Oral mucositis afflicts over 400,000 patients a year in the US, and the incidence is growing as the need for radiation and chemotherapy treatments grows.

Oral mucositis patients are highly susceptible to infection, as a breach in the otherwise protective linings of the oral mucosa and gastrointestinal tract can have serious consequences. The alimentary canal and GI tract are colonized by a vast array of microorganisms, and mucosal lesions can serve as portals of entry for endogenous microorganisms, becoming sites of secondary infection. Although not entirely supported by controlled clinical trials, allopurinol mouthwash and vitamin E have been cited as agents that may decrease the severity of oral mucositis. Prophylaxis against fungal infections is commonly employed in an effort to treat oral mucositis and includes use of topical antifungal agents such as nystatin-containing mouthwashes and clotrimazole troches. Although topical antifungal prophylaxis and treatment may clear superficial oropharyngeal infections, topical agents tend not to be well absorbed and have not been demonstrated to be effective against more deeply invasive fungal infections, which typically involve the esophagus and lower gastrointestinal tract. For this reason, systemic agents are indicated for treating all except superficial fungal infections in the oral cavity.

Treatment of oral mucositis is a significant unmet medical need. Current treatment strategies are primarily palliative, such as cryotherapy (ice chips) to reduce pain and inflammation, and analgesics to manage pain, including mucosal coating mixtures that may contain topical anaesthetics and antibiotics to control the opportunistic infection. These treatments provide little benefit, and do not speed healing or decrease severity of oral mucositis. Agents capable of reducing mucous absorption of the chemotherapy drugs (for example cryotherapy, allopurinol or pilocarpine etc.) have been used, as well as agents which reduce the changes in epithelial proliferation (for example beta-carotene, glutamine or silver nitrate etc.) or anti-inflammatory and antimicrobial agents (for example, mesalazine and/or chlorhexidine). Agents which protect the mucosa (for example, sodium bicarbonate), anaesthetic or analgesic agents (for example, lidocaine, morphine and the derivatives thereof etc.), and agents which accelerate the healing process (for example, vitamin E, tretinoin, laser therapy etc.) or special diets and/or specific oral hygiene regimens have also been employed. The only currently approved therapeutic for oral mucositis is Kepivance™ which is the known mitogenic protein keratinocyte growth factor (KGF) that must be administered intravenously. Kepivance™ is approved for a single indication which comprises only 4% of the total oral mucositis population, i.e., treatment of oral mucositis resulting from pre-conditioning regimens (chemotherapy and radiation) in stem-cell transplant patients. Other compounds have been evaluated for use as a prophylaxis and treatment of oral mucositis. Analgesics such as lidocaine mouthwashes are effective for short periods of time but within hours the pain and discomfort usually returns.

Given that a large number of patients suffer oral mucositis annually and patients undergoing cancer therapy often receive multiple cycles of chemotherapy and/or radiation therapy, there is a significant need for improved treatment of oral mucositis. The present disclosure is directed to this need.

### BRIEF SUMMARY

The following aspects and embodiments thereof described and illustrated below are meant to be exemplary and illustrative, not limiting in scope.

In one aspect, a method of treating oral mucositis is provided, the method comprising topically administering to a subject a therapeutically effective amount of a composition comprising CASAD.

In another aspect, a method to reduce or delay the onset of oral mucositis is provided, the method comprising topically administering to a subject a therapeutically effective amount of a composition comprising CASAD.

In yet another aspect, a method for preventing oral mucositis in a subject is provided, the method comprising topically administering to a subject a therapeutically effective amount of a composition comprising CASAD.

In still another aspect, a method for reducing severity of oral mucositis in a subject is provided, the method comprising topically administering to a subject a therapeutically effective amount of a composition comprising CASAD.

In one embodiment, the CASAD comprises a majority of particles with a dry state fractionation size of between about 50-200 μm. In another embodiment, the CASAD comprises a majority of particles with a wet state fractionation size of less than 2 μm. In a specific embodiment, the CASAD is non-swelling and a calcium species as evidenced by a shrink/swell potential (SV) of less than 1.5 COLE index value. In yet another embodiment, the CASAD exhibits an extractable bases value for calcium of greater than 90 mEq/100 g CASAD, when extracted using ammonium acetate, or a value for calcium of between 6-12 mEq/100 g CASAD/L when extracted with deionized water.

In yet another embodiment, the CASAD has a uniform particle size that is achieved by sieving or air classification.

The method, in one embodiment, is provided to a subject with oral mucositis. In other embodiments, the subject is one at risk of developing oral mucositis.

The method, in another embodiment, is provided to a subject with cancer.

In other embodiments, the method is provided to a subject undergoing or planning to undergo chemotherapy.

In still other embodiments, the method is provided prior to or concurrent with initiation of radiation therapy in the cancer subject.

In yet other embodiments, the method comprises administering after radiation therapy. Alternatively, the method can comprise administering for the duration of radiation therapy.

In other embodiments, the method comprises administering the CASAD composition more than once daily.

In one embodiment, the composition is administered orally as a fluid comprising the CASAD. The fluid can be, for example, a solution, a suspension, a paste, or a gel. In some embodiments, the fluid is held in the mouth for a recommended period of time. In other embodiments, the fluid is swallowed or discharged from the mouth after a recommended period of time.

In other embodiments, the CASAD -containing composition further comprises a polymer. In one embodiment, the polymer is a bioadhesive polymer.

In still other embodiments, the CASAD containing composition comprises a solid dosage form that disintegrates in an aqueous medium. In one embodiment, the aqueous medium is a body fluid.

In other embodiments, the subject receiving the treatment method is concurrently treated with at least one therapeutic agent. In exemplary embodiments, the therapeutic agent is a pain reliever or a chemotherapeutic; an anti-inflammatory or antibiotic. In one embodiment, the pain reliever is a topical anaesthetic selected from the group consisting of fentanyl, hexylresorinol, dyclonine hydrochloride, asbenzocaine and phenol. In a preferred embodiment, the pain reliever is fentanyl.

In one embodiment, the composition for administration is provided in form such that prior to administering, the CASAD is contacted with a fluid to form a composition suitable for oral administration.

Further, the present disclosure includes compositions including the compounds described herein, formulated for administration for reducing the severity of oral mucositis as described herein. As is described in detail below, these compositions can include the compounds in formulations such as gels for topical administration, rinses, tablets, capsules, chewing gum, lozenges, creams, ointments, enemas, suppositories, or patches. In some embodiments, the compositions are administered topically, to a mucous membrane. In some embodiments, the composition is administered orally.

In other aspects, the present disclosure provides methods for treating, ameliorating or preventing oral mucositis comprising administering to a subject a therapeutically effective amount of a composition comprising CASAD. Patients to be treated according to the disclosed methods and compositions include those who have oral mucositis. In addition, patients who do not have, but are at risk of developing, oral mucositis can be treated according to the present disclosure. In the latter group of patients, the treatment can inhibit, delay or prevent the development of oral mucositis. In some embodiments, the patient to be treated is a subject having cancer. In some embodiments, the subject to be treated is suffering from oral mucositis or is at risk of developing oral mucositis. In one embodiment, the patient to be treated has not been and is not currently receiving a vitamin D therapy.

In certain embodiments, the subject has received or will be receiving radiation therapy or chemotherapy. In certain embodiments, the oral mucositis is caused or is likely to result from radiation-induced toxicity in non-malignant tissue. In other embodiments, the oral mucositis is caused or is likely to result from chemical-induced toxicity in non-malignant tissue. In one embodiment, the chemical-induced toxicity is not caused by docetaxel.

In one embodiment, the subject to be treated is a bone marrow transplantation patient. In another embodiment, the subject to be treated is a cancer patient. The subject may have any type of cancer. In certain embodiments, the subject has leukemia, lymphoma, rectal or colorectal cancer, breast cancer, prostate cancer, androgen-dependent prostate cancer, lung cancer, mesothelioma, head and neck cancer, esophageal cancer, gastric cancer, pancreatic cancer, gastrointestinal cancer, renal cell cancer, testicular cancer, germ cell cancer, glioma or any other primary or solid tumor. In one embodiment, the subject does not have androgen-independent prostate cancer.

Examples of treatments that may cause or place a patient at risk of developing oral mucositis are radiation therapy and/or chemotherapy, as described further elsewhere herein or in the background section. Patients that can be treated according to the present disclosure thus include, for example, cancer patients, as well as patients that have recently been, will shortly be, or are currently subject to treatment with head or neck irradiation, or stem cell or bone marrow transplantation.

According to the disclosed methods, compositions used herein can be administered to a patient prior to, concurrently with, or after a treatment that has induced or places the patient at risk of developing oral mucositis, or a combination of these approaches can be used. In an example, the composition is administered at the same time as, within 1-4 hours of, or on the same day as the treatment, and then for 1-3 (e.g., 1-2) days thereafter (e.g., 1-2 times per day). Other examples of treatment regimens are provided below.

The compositions can be administered to patients by any acceptable manner known in the art that achieved topical application to the oral mucosa, such as a gel, rinse, lozenge, cream, ointment, or patch. Also, treatment according to the present disclosure can be carried out in combination with other approaches to treating oral mucositis, including antimicrobial and palliative treatments, as is discussed further below.

In some embodiments, the subject is concurrently treated with at least one therapeutic agent. In some embodiments, the therapeutic is a pain reliever or a chemotherapeutic. In some embodiments, the therapeutic agent is an anti-inflammatory or antibiotic. In some embodiments, the pain reliever is a topical anaesthetic selected from, but not limited to, the group consisting of fentanyl, hexylresorinol, dyclonine hydrochloride, asbenzocaine and phenol. In some embodiments, the administering comprises administering to a subject undergoing or planning to undergo chemotherapy. In some embodiments, the administering is prior to or concurrent with initiation of radiation therapy in the cancer subject. In some embodiments, the administering is after radiation therapy. In some embodiments, the administering continues for the duration of radiation therapy. In some embodiments, the administering continues for longer than the duration of radiation therapy. In some embodiments, the administering continues for a time shorter than the duration of radiation therapy. In some embodiments, the administering comprises administering more than once daily. In some embodiments, the administering comprises administering once daily.

In some embodiments, the composition used in the method does not include vitamin D. In some embodiments, the therapeutic agent is not vitamin D. In some embodiments, the patient is not being or has not been treated with vitamin D.

In addition to the exemplary aspects and embodiments described above, further aspects and embodiments will become apparent by reference to the drawings and by study of the following descriptions.

### BRIEF DESCRIPTION OF THE FIGURES

FIG. 1A shows the mean percentage weight change as a function of day in animals with induced oral mucositis and left untreated (circles) or treated three times daily with the CASAD composition for two different time periods, from study day 0-29 (triangles) or from study day 7-28 (squares);

FIG. 1B is a bar graph showing the area under the curve (AUC) calculated for the percentage weight change in the animals of FIG. 1A;

FIG. 2A is a graph of the mean mucositis score as a function of day, for animals with induced oral mucositis and untreated (circles) or treated three times daily with a CASAD composition for two different time periods, from study day 0-29 (triangles) or from study day 7-28 (squares);

FIG. 2B is a bar graph showing the percent of animals days with a mucositis score of 3 or higher, where the total number of days in which an animal in one of the three treatment groups exhibited score of 3 or higher was summed and expressed as a percentage of the total number of days scored. Statistical significance was evaluated using the Chi-squared test; and

FIGS. 3A-3B are bar graphs showing the amount, in pg/mL of TNF-a present in solution after incubation with the noted concentrations of CASAD (FIG. 3A) and the percent of TNF-a relative to the control solution containing no clay for each of the CASAD solutions (FIG. 3B).

Various aspects now will be described more fully hereinafter. Such aspects may, however, take many different forms and should not be construed as limited to those embodiments explicitly set forth herein. Rather, these embodiments are provided so that this disclosure will be thorough and complete, and will fully convey its scope to those skilled in the art.

### DETAILED DESCRIPTION

### I. Definitions

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

It must be noted that, as used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “oral mucositis” intends inflammation and/or destruction of the mucosal epithelium lining of the mouth cavity, including the cheeks, gums, tongue, lips, the roof or floor of the mouth.

As used herein, the phrase “therapeutically effective amount” refers to an amount which provides a therapeutic benefit, wherein benefits can include, the prevention, treatment or amelioration of mucositis.

### II. Methods of Treatment

In a first aspect, a method for preventing, treating, ameliorating and/or reducing the severity of oral mucositis is provided. As will be illustrated by the studies discussed herein, administration of a composition comprising CASAD is effective in treating, slowing the progression of, reducing the severity of, and preventing mucositis. These studies are described in Section A. In Section B, formulations comprising CASAD for use in the treatment method are described, and in Section C, treatment regimens in general and for particular patient populations are disclosed.

### A. Treatment of Oral Mucositis

Studies were conducted in support of the claimed methods to demonstrate the efficacy of a composition comprising CASAD to prevent oral mucositis, treat oral mucositis, ameliorate the severity of oral mucositis and/or reduce the severity of oral mucositis. As described in Example 1, an acute radiation model was used wherein oral mucositis was induced in the buccal pouch of hamsters. In the study, an acute radiation dose of 40 Gy was administered to each animal. Clinically significant oral mucositis was observed on days 12 through 28.

A composition comprising CASAD was given topically to the left cheek pouch three times daily either from Day 0 to Day 28 (Group 2) or from Day 7 to 28 (Group 3). As a control, animals in Group I received a saline vehicle three times daily at the same dose volume as the CASAD compositions. The CASAD selected for use in the study was a calcium-based aluminosilicate clay, which is essentially non-swelling in aqueous fluids. The CASAD is discussed in more detail hereinbelow.

Oral mucositis in the left cheek pouch for the treated and control animals was evaluated clinically starting on Day 6, and continuing on alternate days until Day 28. The weight and general health of the animals were evaluated daily. Beginning on day 6 and continuing on alternate days for the duration of the study, oral mucositis was scored using a standard six point scale, set forth in Example 2 below. The number of days of ulcerative mucositis was evaluated using a chi-squared test of oral mucositis scores of 3 or higher, and the individual daily group scores were assessed with a rank sum test.

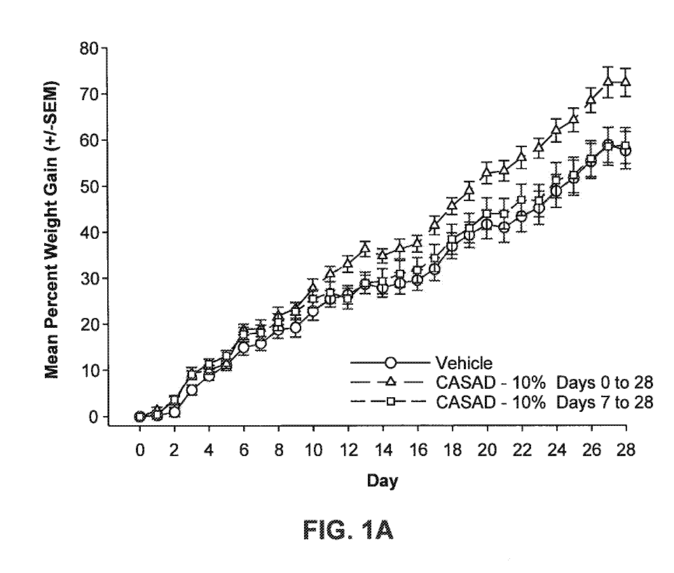

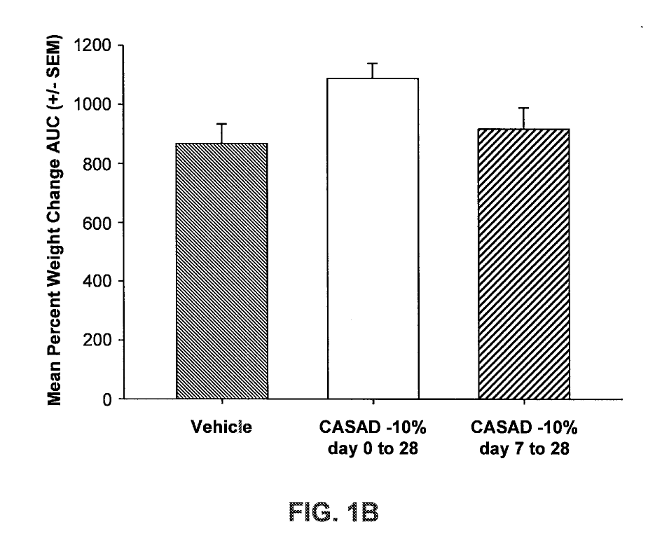

The mean daily percent weight change of the test animals is presented in FIG. 1A. The saline vehicle-treated control hamsters (circles) gained an average of 51.9% of their starting weight during the study. Hamsters in the group treated with 10% CASAD from Day 0 to 28 (triangles) gained an average of 59.5% of their starting weights during the study. Hamsters in the group treated with 10% CASAD from Day 7 to 28 (squares) gained an average of 62.7% their starting weights during the course of the study. It is interesting to note that the 10% CASAD from Day 0 to 28 treatment group showed no difference in weight gain from the other groups until Day 10 when the oral mucositis begins to becomes significant. After Day 10 the Day to 28 CASAD group gained substantially more weight than either the vehicle control group of the group treated with CASAD from Day 7 to 28.

The statistical significance of these differences was evaluated by calculating the area-under-the-curve (AUC) using the trapezoidal rule transformation for the weight gain of each animal, and then comparing the different treatment groups using a One-Way ANOVA test. The results of this analysis indicated that there were no significant differences between any of the treatment groups (p=0.056). These data are shown in FIG. 1B.

The grade of oral mucositis was scored, beginning on day 6, and for every second day thereafter, through and including day 28. The effect on oral mucositis of each drug treatment compared to placebo was assessed. On each evaluation day, the number of animals with a blinded oral mucositis score of 3 in each drug treatment group was compared to the control group. Differences were compared on a cumulative basis and statistical significance was determined by chi-square analysis. Efficacy in this analysis is defined by a significant reduction in the number of days that a group of animals had ulcerations (scores 3) when compared to the control group.

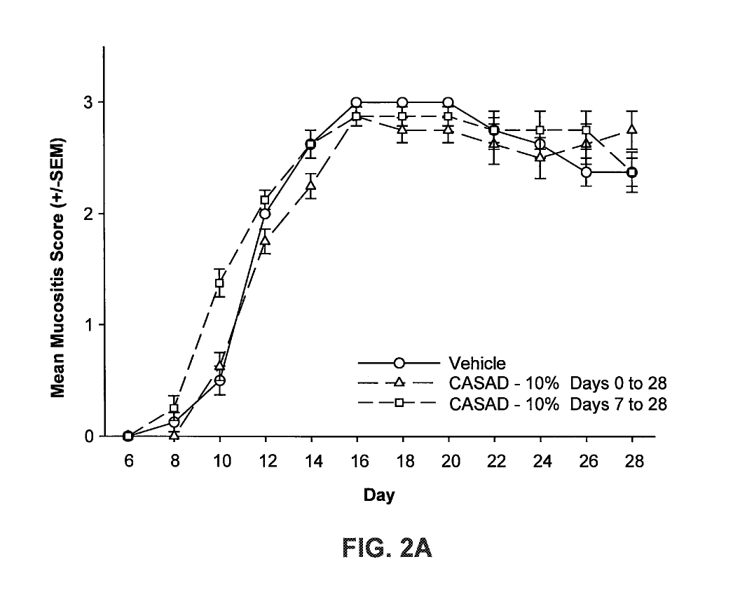

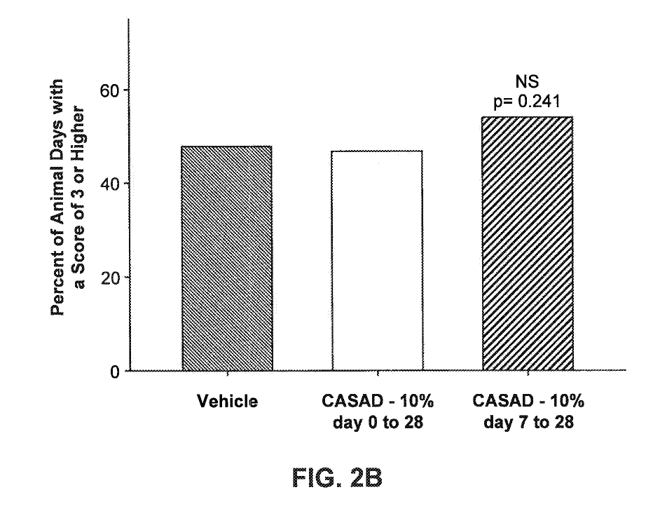

The results of this analysis are shown in Table 1 and FIGS. 2A-2B.

TABLE 1
X² analysis of the total number of days the animals in each group
spent with a score of three or more. This statistic is a measure of severity
of ulceration, a clinically important outcome.

| | | | Total | | Chi Sq | |
|---|---|---|---|---|---|---|
| | Days | Days | Animal | % Days | vs. | |
| Treatment Group | ≧3 | <3 | Days | ≧3 | Control | P Value |
| 1 | 92 | 100 | 192 | 47.92% | — | — |
| (Vehicle Control) | | | | | | |
| 2 | 90 | 102 | 192 | 46.88% | 0.0104 | p = 0.919 |
| (10% CASAD, | | | | | | |
| Day 0 to 28) | | | | | | |
| 3 | 104 | 88 | 192 | 54.17% | 1.261 | p = 0.261 |
| (10% CASAD, | | | | | | |
| Day 7 to 28) | | | | | | |

In the vehicle control group, the percentage of animal days with a score of 3 or higher was 47.9%. In the group treated with 10% CASAD from Day 0 to 28, the percentage of animal days with a score of 3 or higher was slightly reduced, to 46.9%. In the group treated with 10% CASAD from Day 7 to 28, the percentage of animal days with a score of 3 or higher was increased, up to 54.2%.

An analysis of the severity of oral mucositis was performed using the Mann-Whitney rank sum analysis to compare the scores for each treatment group to the controls on each day of the analysis. In this analysis, 2 days of significant reduction in the oral mucositis score are generally required before it is regarded as meaningful. The p values for each calculation are shown. The results of this analysis are shown in Table 2. Significant improvements are shown underlined and in bold, while significant worsening of disease is shown underlined in italics and underlined.

TABLE 2
A Comparison of Daily Oral Mucositis Scores.

| Group | Treatment Day | | | | | | | | | | | |
|---|---|---|---|---|---|---|---|---|---|---|---|---|
| Comparison | 6 | 8 | 10 | 12 | 14 | 16 | 18 | 20 | 22 | 24 | 26 | 28 |
| Group 1 vs. Group 2 | 1.000 | 0.164 | 0.497 | 0.038 | 0.037 | 0.164 | 0.038 | 0.038 | 0.862 | 0.808 | 0.107 | 0.018 |
| (vehicle vs. CASAD- | | | | | | | | | | | | |
| Day 0 to 28) | | | | | | | | | | | | |
| Group 1 vs. Group 3 | 1.000 | 0.388 | <0.001 | 0.164 | 0.982 | 0.164 | 0.164 | 0.164 | 0.524 | 0.204 | 0.018 | 0.816 |
| (vehicle vs. CASAD- | | | | | | | | | | | | |
| Day 7 to 28) | | | | | | | | | | | | |

The group treated with 10% CASAD from Day 0 to 28 showed significant reductions in oral mucositis scores relative to vehicle controls on four separate days of the study (Days 12, 14, 18, and 20) while those where treatment started later, on day 7, did not exhibit any days of significant improvement. Interestingly, there was a trend for significant worsening of oral mucositis as the disease begins to resolve between Days 26 and 28 in those animals treated with CASAD. This suggests that dose scheduling studies may play a role in determining the most effective timing of treatment with the CASAD-containing composition.

Treatment of the animals with a CASAD-containing composition was effective in reducing the severity of oral mucositis when dosed from Day 0 to 28. Importantly, the CASAD treatment resulted in a substantial reduction in percent of animals with ulcerative oral mucositis during peak disease from Days 14 to 20.

Treatment with 10% CASAD from Day 0 to 28 significantly reduced the daily oral mucositis scores on four of the twelve days evaluated when compared to vehicle control. Treatment with 10% CASAD from Day 0 to 28 reduced the percent of animals with ulceration compared to vehicle control by 37.5% on Day 14, by 12.5% on Day 16, and by 25% on Days 18 and 20.

Oral mucositis typically progresses in five phases. Phase 1, “the initial phase,” includes: DNA strand breaks, and reactive oxygen species generation. Phase 2, “the primary damage response phase” includes: activation of NFκB and p53 pathway; NFκB up-regulation of genes that may exert an effect on mucosal toxicity, including apoptosis-regulating genes of the BCL2 family; up-regulation of c-Jun and c-Jun amino-terminal kinase, which in turn up-regulates NRF2; and production of proinflammatory cytokines, TNF-alpha, IL-1β, IL-6, the presence of which may cause damage to epithelium via reduced oxygenation and basal cell death, endothelium, and connective tissue; radiation and some cytotoxic agents also cause apoptosis via hydrolyzation of sphingomyelin (a cell-membrane lipid), a process that increases ceramide levels and results in cell apoptosis; fibroblasts in the submucosa may be damaged by radiation or chemotherapy, either directly or via stimulation of metalloproteinases. Phase 3, “the signal amplification phase,” includes: a range of proteins that accumulate and target the submucosa, causing tissue damage and initiating a positive feedback loop, amplifying the primary damage caused by the radiation or chemotherapy. For example, a pathway that results in cell death is activated by TNF-alpha, which in turn activates NFκB and initiates mitogen-activated protein kinase (MAPK) signaling, in turn activating JNK (a member of the MAP kinase family), in turn regulating the activity of AP 1. Cell death caused by this pathway occurs in the submucosa as well as the epithelium. TNF-alpha and IL-1beta. both induce matrix metalloproteinase activation. Phase 4, “the ulcerative phase,” may include: functional trauma caused lesions (e.g., with respect to oral mucositis, the lesions appear in the mouth); excessive bacterial colonization of lesions, (e.g., with respect to oral mucositis, the bacterial colonization of lesions may be exacerbated by reduced salivary levels and poor oral hygiene as often happens in neutropenic patients); endotoxin released from gram-negative organisms and cell wall products from gram-positive bacteria may then interact with tissue macrophages to trigger release of further IL-1 and TNF-alpha, exacerbating mucosal damage. Secondary infections that result include fungal infections, viral infections and bacterial infections. Phase 5, “the healing phase,” includes cell proliferation and differentiation returns to normal; bone marrow recovery results in increased numbers of white cells and control of local infection.

In another study conducted in support of the claimed methods, the binding of cytokines to the calcium aluminosilicate clay was investigated. In this study, a fixed concentration of cytokine was exposed to varying concentrations of CASAD for 15 minutes at room temperature. The samples were stirred occasionally and then centrifuged. The concentration of protein was determined using a sandwich immunoassay kit (R&D Systems, Minneapolis, Minn.) and compared to that measured in a sample without CASAD. Results are shown in Table 3.

TABLE 3

CASAD
Concentration
(mg/mL)

| | | | |
|---|---|---|---|
| 0.01 | 0.1 | 1.0 | 10.0 |

| | | |
|---|---|---|
| | Concentration | Percent of Cytokine |
| Material | (pg/mL) | Bound by CASAD |

| | | | | | |
|---|---|---|---|---|---|
| IL-1β | 750 | 40 | 52 | 71 | 80 |
| IL-6 | 750 | 35 | 41 | 45 | 89 |
| IL-8 | 200 | 0 | 29 | 43 | ND |
| TNF-α | 750 | 5 | 61 | 90 | 99 |

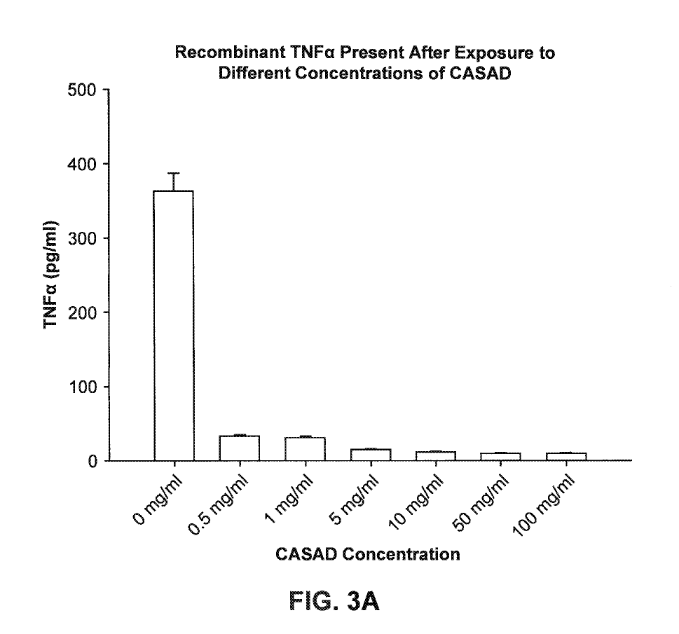

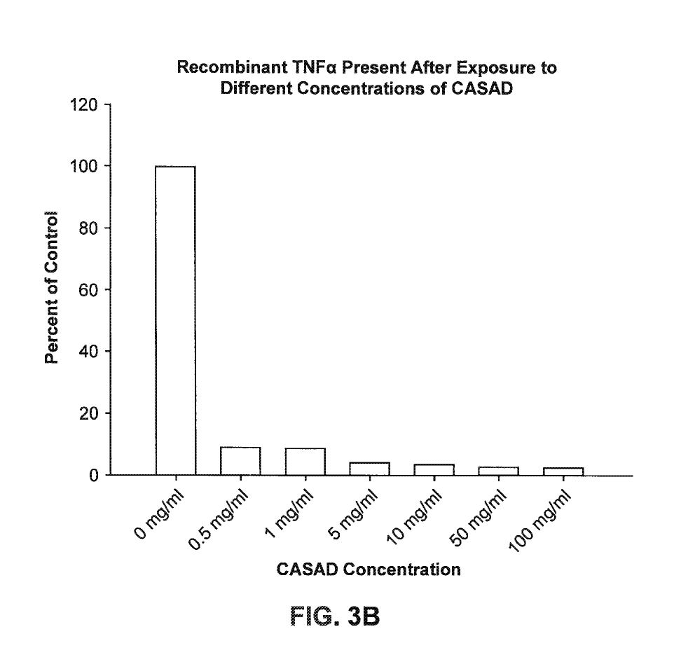

The data in Table 3 shows that pro-inflammatory cytokines such as TNF-alpha are readily bound by the CASAD. At a concentration of 1 mg/mL CASAD, 90% of the TNF-alpha was removed from solution presumably by direct binding to the CASAD. Binding IL-1beta by the CASAD was moderate while binding of IL-6 and IL-8 was weak. The unique ability of the CASAD to bind TNF-alpha was further investigated, as blocking the action of TNF can be beneficial in reducing the inflammation in mucositis and in other diseases. The study is described in Example 3, and the data is presented in FIGS. 3A-3B. The data presented in the two graphs demonstrates the removal of TNF-alpha from a stock solution of this pro-inflammatory protein. Even concentrations of the CASAD as low as 0.5 mg/ml removed greater than 90% of all TNF-a from solution. The study demonstrates the capacity to bind and remove from solution important inflammatory components such as tumor necrosis factor and, also illustrates the small amount of CASAD needed to achieve a benefit. The later type of information is of direct relevance in determining appropriate doses for CASAD compositions.

Accordingly, in one embodiment a composition comprising CASAD to prevent oral mucositis, treat oral mucositis, ameliorate the severity of oral mucositis and/or reduce the severity of oral mucositis is provided to a subject in need. The ability of the CASAD composition to alter the pro-inflammatory environment in the tissue area is demonstrated by the data, and the efficacy of the CASAD composition to prevent and ameliorate oral mucositis is evident from the animal studies.

### B. CASAD and Formulations Comprising CASAD

The methods of treatment comprise providing to a patient, for administration by a suitable route depending on the extent of tissue damage and condition of the patient, a composition comprising the CASAD.

### B1. Clay

Clays are a distinguished from other fine-grained earth deposits, and are grouped distinct classes such as kaolinites, illites, smectites. Clays based on silicates form a large class, where the basic structural unit for silicate clays is a SiO4 tetrahedron in which Si⁴⁺ is located at the center and four O²⁻ are positioned at the apices. The tetrahedral structures can be linked together by sharing four O²⁻ ions and together can form a variety of more complex structures including rings (cyclosilicates), chains (inosilicates), sheets (phyllosilicates) and three dimensional arrangements (tectosilicates). Tetrahedra, e.g., SiO₄ and octahedral, e.g., Al₂O₃ are common structural components in many mineral structures.

The clay for use in the methods described herein is a dioctahedral smectite, calcium aluminosilicate clay. Calcium aluminosilicate clay is a 2:1 phyllosilicate clay containing sheets of 6-membered rights, and is a very pure calcium montmorillonite clay in the dioctahedryl smectite group. Its general chemical formula is (Na, Ca)_{0.3}(Al, Mg)₂Si₄O₁₀(OH)₂ n(H₂). An exemplary non-swelling calcium clay is described in U.S. Patent Application Publication No. 2008/0026079, which is incorporated by reference herein.

This calcium aluminosilicate clay is a ‘non-swelling’ clay due to the fact it contains more calcium than sodium. As used herein, essentially “low-swelling” and essentially “non-swelling” clay species means that the particular clay species has minimal capacity for changes in volume due to shrinkage or swelling. One approach to measure swelling potential is given in the Soil USDA Technical Handbook, where a COLE index value is assigned to materials. A COLE index value >0.03 indicates that a material may have a small amount of smectite clay within its composition and show low shrink/swell potential. Sodium montmorillonite clays swell much more in aqueous fluids than calcium montmorillonte clays. In one embodiment, the clay for use in the methods described herein is a montmorillonite clay that is non-swelling and a calcium species, as evidenced by a shrink/swell potential (SV) of less than 1.5 COLE index value.

Due to the presence on the surface of many functional groups, clays are able to interact with other components of the formulation. Negative charges on the clay particles are compensated by counterions, for example Na+, Ca++, Ag+, and so forth, or a combination thereof. Cation exchange capacity (CEC) is an intrinsic property defining the concentration of negatively charged sites on clay particles. Cation exchange capacity is a measure of exchangeable bases in the clay material, and provides an indication of the capacity of the clay to exchange/interact with other compounds. The cationic exchange capacity of clays can be measured using the method described in Grimshaw, The Chemistry and Physics of Clays, Interscience Publishers, Inc., pp. 264-265 (1971). A common method for determining CEC uses 1 M ammonium acetate (NH₄OAc) at pH 7 (neutral NH₄OAc) and is a standard method used for soil surveys by the Natural Resource Conservation Service. In one embodiment, the clay for use in the methods described herein exhibits an extractable bases value for calcium of greater than 90 mEq/100g clay, when extracted using ammonium acetate, or a value for calcium of between 6-12 mEq/100 g clay/L when extracted with deionized water.

A skilled artisan will appreciate that clay can be sized by sieving or by air classification to achieve a desired average particle size. A skilled artisan in the relevant field will appreciate that any representative sample of clay will be polydisperse in size, yet the art provides several approaches for expressing the average size, or diameter, of particles in a population. For example, an average particle size, or uniform particle size, may in some embodiments intend that a representative sample of the clay when passed through a sieve of a certain mesh size retains a majority (greater than 50%) of the clay sample. Size fractionation (wet and dry) of a representative sample of a given clay can be done as follows. Wet state fractionation involves removal of cementing and flocculating materials in the sample using sodium acetate buffer (pH 5). Organic matter in the sample is removed using 30% hydrogen peroxide. The sample is then dispersed with 50 mL of pH 10 sodium carbonate solution. The sand fraction (>53 μm) is separated using a 53 μm sieve. The clay fraction (<2 μm) is separated from the silt fraction (2-53 μm) by centrifugation using pH 10 sodium carbonate as dispersant. Sand and silt weights are recorded after drying at 105° C. overnight. Clay suspension is flocculated with sodium chloride and then dialyzed until the electrical conductivity measurement were close to the values of deionized water (<2 μS/cm). Air dry fractionation of clay can be done using, for example, an Octagon 200 sieve shaker (Endecotts), and weight average particle size determined in accord with the manufacturer's instructions. For example, the percentage of particles with greater than 100 μm size, between 45-100 μm size, and less than 45 μm size is calculated. In one embodiment, the montmorillonite clay comprises a majority (i.e., 51%, based on weight percent) of particles with a dry state fractionation size of between about 50-200 μm. In other embodiments, the montmorillonite clay is comprised of a particles wherein at least 60%, 70%, 75% or 80% of the particles have a dry state fractionation size of between about 50-200 μm. In another embodiment, montmorillonite clay comprises a majority of particles with a wet state fractionation size of less than 2 μm. In another embodiment, the average particle size of the clay in a dry state is less than about 200 μm, or less than about 100 μm, or less than about 80 μm. In other embodiments, the average particle size of the clay is between 5-200 μm or between 5-50 μm.

### B2. Formulations Comprising CASAD

The CASAD is formulated with one or more pharmaceutical excipients to provide a composition for administration to a patient in need of treatment. Compositions contemplated for use in the methods are varied according to these, and other, factors, and exemplary formulations are now described.

In a first embodiment, a composition suitable for topical oral administration to the mucosal tissue in the oral cavity of a patient in need is contemplated. The composition comprises an amount of CASAD sufficient to achieve a desired therapeutic response when administered to the patient in accord with a defined protocol. Formulation of the CASAD into a form suitable for oral administration includes formulation to provided, for example, a liquid formulation such as a suspension, a solution, an elixir, or an emulsion, that can be used as a mouth rinse, spray or wash. Liquid formulations often are aqueous based and can include excipients to increase the viscosity to provide a coating on the mucosal tissue that lingers for a period of time after application. Mouthwash formulations are well-known to those skilled in the art and will often include excipients as, for example, pharmaceutical grades of man nitol, lactose, starch, magnesium stearate, sodium saccharine, cellulose, magnesium carbonate and/or the like. Exemplary formulations are discussed in detail, for example, in U.S. Pat. No. 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, each of which is herein specifically incorporated by reference into this section of the specification and all other sections of the specification.

Oral formulations can also take the form of a lozenge, a treated substrate such as a topical swab or pad, that comprises the CASAD, or a buccal patch or other bioadhesive polymeric compositions comprising the CASAD. Lozenges are typically discoid-shaped solids containing the CASAD in a suitably flavored base. The base may be a hard sugar candy, glycerinated gelatin, or the combination of sugar with sufficient gelatin to give it form. Lozenges are placed in the mouth where they slowly dissolve, liberating the CASAD for direct contact with the affected mucosa. Lozenges can be prepared, for example, by adding water slowly to a mixture of the powdered CASAD along with excipients such as a sugar and a gum until a pliable mass is formed. The mass is rolled out and the lozenge pieces cut from the flattened mass, or the mass can be rolled into a cylinder and divided. Each cut or divided piece is shaped and allowed to dry, to thus form the lozenge dosage form.

Bioadhesive compositions are particularly suitable for treating oral mucositis. Buccal patches comprising a polymer and the CASAD, where the polymer becomes adhesive in the presence of saliva, are known in the art. Nonlimiting examples of biocompatible polymers that can be used to make a bioadhesive composition include polyethers, such as polyoxyalkylene block copolymers; cellulosic polymers (including hydroxypropylmethyl cellulose, hydroxymethyl cellulose, hydroxypropyl cellulose, methyl cellulose and ethylhydroxyethyl cellulose); gelatin; polyethylene glycol; polyacrylic acid (such as Carbopol™ gel); and glycerol (glycerin). More than one of these exemplary polymers may be included in the composition to provide the desired characteristics and other biocompatible polymers or other additives may also be included in the composition to the extent the inclusion is not inconsistent with performance requirements of the composition.

### Optional Additives

The composition may also include conventional additives such as adhesive agents, antioxidants, crosslinking or curing agents, pH regulators, pigments, dyes, refractive particles, conductive species, antimicrobial agents, active agents and permeation enhancers. In those embodiments wherein adhesion is to be reduced or eliminated, conventional detackifying agents may also be used. These additives, and amounts thereof, are selected in such a way that they do not significantly interfere with the desired chemical and physical properties of the composition.

Non-limiting examples of suitable excipients, diluents, and carriers include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; carriers such as propylene glycol and ethyl alcohol, and lubricants such as talc, calcium and magnesium stearate, and solid polyethyl glycols.

Additives may be present in the compositions, such as flavoring, sweetening or coloring agents, or preservatives. Mint, such as from peppermint or spearmint, cinnamon, eucalyptus, citrus, cassia, anise and menthol are examples of suitable flavoring agents. Flavoring agents are preferably present in the oral compositions in an amount in the range of from 0 to 3%; preferably up to 2%, such as up to 0.5%, preferably around 0.2%, in the case of liquid compositions. Sweeteners include artificial or natural sweetening agents, such as sodium saccharin, sucrose, glucose, saccharin, dextrose, levulose, lactose, mannitol, sorbitol, fructose, maltose, xylitol, thaumatin, aspartame, D-tryptophan, dihydrochalcones, acesulfame, and any combinations thereof, which may be present in an amount in the range of from 0 to 2%, preferably up to 1% w/w, such as 0.05 to 0.3% w/w of the oral composition.

Other optional ingredients of oral aqueous compositions include humectants, surfactants (non-ionic, cationic or amphoteric), thickeners, gums and binding agents. A humectant adds body to the mouthspray formulation and retains moisture in a dentifrice composition. In addition, a humectant helps to prevent microbial deterioration during storage of the formulation. It also assists in maintaining phase stability and provides a way to formulate a transparent or translucent dentifrice. Suitable humectants include glycerine, xylitol, glycerol and glycols such as propylene glycol, which may be present in an amount of up to 50% w/w each, but total humectant is preferably not more than about 60-80% w/w of the composition. For example, liquid compositions may comprise up to about 30% glycerine plus up to about 5%, preferably about 2% w/w xylitol.

When the oral compositions are in the form of a mouthspray, it is preferred to include a film forming agent up to about 3% w/w of the oral composition, such as in the range of from 0 to 0.1%, preferably about 0.001 to 0.01%, such as about 0.005% w/w of the oral composition. Suitable film-formers include (in addition to sodium hyaluronate) those sold under the tradename Gantrez™.

### C. Exemplary Dosing Regimens and Patient Populations

In the studies conducted herein, the CASAD composition was applied topically to the mucosal tissue in the oral cavity three times daily. It will be appreciated that this dosing regimen is merely exemplary, and the dosing schedule can be varied according to each individual, to the severity of oral mucositis and in accord with other parameters. By way of example, the CASAD formulation can be applied topically to mucosal surfaces of the oral cavity, in some embodiments, one to eight applications per day can begin 24 hours before chemotherapy or radiation, and may continue after the conclusion of cancer treatment or other therapy associated with mucositis. By way of another example, the CASAD formulation can be administered to the desired local area, one, two, three, four, five or more times per day, and treatment can begin before or concurrent with chemotherapy and/or radiation, and may cease prior to the end of chemotherapy and/or radiation or may continue after chemotherapy and/or radiation has ended.

In one embodiment, the method is for treating or preventing oral mucositis resulting from radiation or chemotherapy for cancer. The method includes the step of administering to a patient an effective amount of a solution or suspension containing the therapeutic CASAD. The solution or suspension is administered as, for example, a mouth-rinse.

In another embodiment, the method for treating or preventing oral mucositis resulting from radiation or chemotherapy for cancer includes the step of administering a solid dosage form to the oral cavity of a patient, for example, sublingually, wherein CASAD comes into contact with the inflamed tissue. The solid dosage form is one intended to be retained in the oral cavity and not ingested or swallowed by the patient.

Treatment according to the disclosed methods can begin prior to cancer treatment or other condition or therapy associated with oral mucositis (e.g., propylactically, and/or 1-2 days or up to 1 week prior), at or near the same time as cancer treatment or other therapy associated with oral mucositis (e.g., simultaneously with, within 1-4 hours of, or on the same day as the treatment), or shortly after the cessation of cancer treatment or other condition or therapy associated with oral mucositis (e.g., within 1-4 days of cessation, and/or prior to or upon appearance of symptoms). Treatment can then be maintained, for example, until any symptoms of oral mucositis have substantially cleared or the risk of developing such symptoms has passed. Thus, treatment started before or at or near the same time as cancer treatment or other condition or therapy associated with oral mucositis can be maintained, e.g., for 1-3, e.g., 1-2 days. In other examples, treatment is maintained for 1-4 or 2-3 weeks following the cessation of cancer treatment or other therapy associated with oral mucositis, as determined to be appropriate by one of skill in the art. In specific examples, the treatment according to the present disclosure is carried out only prior to cancer treatment or other therapy associated with oral mucositis; prior to and concurrently with cancer treatment or other therapy associated with oral mucositis; prior to, concurrently with, and after cessation of cancer treatment or other therapy associated with oral mucositis; concurrently with cancer treatment or other therapy associated with oral mucositis only; concurrently with and after cessation of cancer treatment or other therapy associated with oral mucositis only; after cessation of cancer treatment or other therapy associated with oral mucositis only; or prior to and after cessation of cancer treatment or other therapy associated with oral mucositis only. Further, treatment according to the methods of the present disclosure can be altered, stopped, or re-initiated in a patient, depending on the status of any symptoms of oral mucositis. Treatment can be carried out at intervals determined to be appropriate by those of skill in the art. For example, the administration can be carried out 1, 2, 3, 4 or more times/day. It will be appreciated that the patients to be treated with the methods described herein are not limited to cancer patients, but include any patient that is at risk of or has developed oral mucositis.

Chemotherapeutic agents likely to cause oral mucositis include but are not limited to anthracyclines (such as daunorubicin, doxorubicin, pirubicin, idarubicin and mitoxantrone), methotrexate, dactinomycin, bleomycin, vinblastine, cytarabin, fluorouracil, mitramycine, etoposide, floxuridine, 5-fluorouracil, hydroxyurea, methotrexate, mitomycin, vincristine, vinorelbine, taxanes (such as docetaxel and paclitaxel), ifosfamide/eoposide, irinotecan, platinum, as well as combinations including one or more of these drugs. The risk of developing oral mucositis is markedly exacerbated when chemotherapeutic agents that typically produce mucosal toxicity are given in high doses, in frequent repetitive schedules, or in combination with ionizing irradiation (e.g., conditioning regimens prior to bone marrow transplant). The lesions induced by chemotherapeutic agents are clinically significant by about a week after treatment and the severity progresses to about day ten through twelve and begins to subside by day fourteen. Accordingly, in some embodiments, the patient to be treated is one undergoing or scheduled to undergo treatment with one or more of these chemotherapeutic agents.

Non-therapeutic radiation and/or chemical exposure, as may happen from accidents, acts of war, acts of civilian terrorism, space flights, or rescue and clean-up operations can also result in oral mucositis. In these scenarios the effects of radiation in the hematopoietic system and the gastrointestinal tract are critical. Furthermore, inflammation can be caused by conditions in the mouth itself, such as poor oral hygiene, dietary protein deficiency, poorly fitted dentures, or from mouth burns from hot food or drinks, toxic plants, or by conditions that affect the entire body, such as medications, allergic reactions, radiation therapy, or infections.

### Combination Regimens and Combination Compositions

The methods presently disclosed can be used alone or in conjunction with other approaches to reducing the severity of oral mucositis. For example, the disclosed methods can be carried out in combination with antimicrobial or antifungal therapies, e.g., therapies involving administration of antibiotics such as nystatin, amphotericin, acyclovir, valacyclovir, clotimazole, and fluconazole. As a specific example of such treatment, patients with head and neck cancer receiving radiotherapy have colonization of the oropharyngeal region with gram-negative bacteria. Selective decontamination of the oral cavity with anti-microbial agents has the benefit of reducing oral mucositis associated with radiation therapy, but there may be limitations to the beneficial effects of such treatment. Anti-microbial therapy can kill bacteria, but cannot reduce endotoxin, and indeed may actually increase endotoxin. As endotoxin is a potent mediator of inflammation, it may contribute to the aggravation of mucositis and, thus, co-treatment with an antiendotoxin compound (e.g., a Lipid A analog, such as eritoran) and antibiotics can be used as a more effective approach to treating oral mucositis in such patients, according to the present disclosure.

The methods presently disclosed can also be used in conjunction with palliative therapies including the use of topical rinses, gels, or ointments that include lidocaine, articaine, and/or morphine, as well as other analgesic or anti-inflammatory agents. Specific examples of other agents and approaches that can be used in combination with TLR4 antagonists, according to the methods presently disclosed, include the following: palifermin (recombinant keratinocyte growth factor; rHuKGF; Kepivance™; Amgen) and AES-14 (uptake-enhanced L-glutamine suspension) (Peterson, J. Support Oncol. 4(2 Suppl. 1)9-13, 2006); oral cryotherapy, low-level laser therapy, chlorhexidine, amifostine, hematologic growth factors, pentoxifylline, and glutamine (Saadeh, Pharmacotherapy 25(4):540-554, 2005); amifostine, antibiotic paste or pastille, hydrolytic enzymes, ice chips, benzydamine, calcium phosphate, honey, oral care protocols, povidone, and zinc sulphate (Worthington et al., Cochrane Database Syst. Rev. 2:CD000978, 2006); flurbiprofen (e.g., administered as a tooth patch; Stokman et al., Support Care Cancer 13(1):42-48, 2005); diphenhydramine, magnesium hydroxide/aluminum hydroxide, nystatin, and corticosteroids (Chan et al., J. Oncol. Pharm. Pract. 11(4):139-143, 2005); oral transmucosal fentanyl citrate (e.g., administered in the form of a lozenge; Shaiova et al., Support Care Cancer 12(4):268-273, 2004); clonazepam (e.g., in the form of a tablet; Gremeau-Richard et al., Pain 108(102):51-57, 2004); capsaicin (e.g., in the form of a lozenge; Okuno et al., J. Cancer Integr. Med. 2(3):179-183, 2004); ketamine (e.g., in the form of an oral rinse; Slatkin et al., Pain Med. 4(3):298-303, 2003); and granulocyte-macrophage colony-stimulating factor (GM-CSF)/granulocyte colony-stimulating factor (G-CSF), laser light therapy, and glutamine supplements (Duncan et al., Aliment. Pharmacol. Ther. 18(9):853-874, 2003).

An exemplary assay for the treatment of oral mucositis may be performed as described in the phase 3 clinical trial of Kepivance^{TM} (palifermin) (see, Spielberger, N. Engl. J. Med., 351(25):2590-2598 (2004)), or as described in phase II clinical trials of GM-CSF (molgramostin) (see McFleese et al., Br. J. Radiol. 79(943):608-13 (2006)).

In some embodiments, the patient is not undergoing, or has not undergone treatment with vitamin D.

In some embodiments of the present disclosure, the CASAD may be combined with a second therapeutic agent. In one embodiment, the second therapeutic agent is a pain reliever or anaesthetic, such as an anaesthetic found in a lozenge, spray or mouth rinse (e.g., phenol, benzocaine, phenazone, antipyrine, analgesine, dyclonine hydrochloride salt). In some embodiments, the pain reliever acts as a bactericidal and fungicidal in addition to acting as a local anaesthetic. In some embodiments, the pain reliever is an anaesthetic selected from the group consisting of fentanyl, hexylresorinol, dyclonine hydrochloride, asbenzocaine and phenol. For example, fentanyl has been approved for topical administration (transdermal patch), and is often used in cancer for pain control. In some embodiments, the CASAD is combined with fentanyl.

The Toll-like receptor (TLR) family plays a fundamental role in pathogen recognition and activation of innate immunity. Toll-like receptor 4 is a protein that in humans is encoded by the TLR4 gene. The TLR4 receptor detects lipopolysaccharide (LPS) from Gram-negative bacteria and is thus important in the activation of the innate immune system. TLR4 has also been designated as CD284 (cluster of differentiation 284). Examples of TLR4 agonists are: morphine, morphine-3-glucuronide, oxycodone, levorphanol, pethidine, fentanyl, methadone and buprenorphine.

According to the methods disclosed herein, a TLR4 antagonist can be administered to a patient before, during, and/or after treatment with a therapy that causes oral mucositis or puts the patient at risk of developing oral mucositis. As is noted above, such treatments include radiation and chemotherapy, which act by blocking the growth of rapidly dividing cells, such as cancer cells and epithelial cells that line the surfaces of the oral cavity. Specific examples of treatments that can lead to oral mucositis include radiation treatment (e.g., head and/or neck, whole body, targeted, and/or hyperfractionated radiation), as well as chemotherapeutic regimens used in the treatment of, or as adjuvant treatments for, conditions such as breast cancer, colon cancer, gastric cancer, genitourinary (e.g., bladder, prostate, or testicular) cancer, gynecologic (e.g., cervical, endometrial, ovarian, or uterine) cancer, head and neck/esophageal cancer, leukemia, lung (small cell or non small-cell) cancer, lymphoma (Hodgkin's or non-Hodgkin's), melanoma, multiple myeloma, pancreatic cancer, and sarcoma.

### EXAMPLES

The following examples are given to illustrate the present disclosure. It should be understood that the claims are not to be limited to the specific conditions or details described in the examples.

### Example 1

### Acute Radiation Model of Mucositis

An acute radiation model in hamsters has proven to be an accurate, efficient and cost-effective technique to provide a preliminary evaluation of anti-mucositis compounds (Murphy, C. K. et al., (2008) Clin Cancer Res. 1(14):4292-7). The course of mucositis in this model is well-defined and results in peak scores approximately 14-16 Days following radiation. The acute model has little systemic toxicity, resulting in few hamster deaths, thus permitting the use of smaller groups (N=7-8) for initial efficacy studies. It has also been used to study specific mechanistic elements in the pathogenesis of mucositis. Molecules that show efficacy in the acute radiation model may be further evaluated in the more complex models of fractionated radiation, chemotherapy, or concomitant therapy.

### Animals

Male LVG Syrian Golden Hamsters (Charles River Laboratories), aged 5 to 6 weeks, with average body weight of 89.5 g at study commencement, were used. Animals were individually numbered using an ear punch and housed in small groups of approximately 8 animals per cage. Animals were acclimatized prior to study commencement. During this period of 3 days, the animals were observed daily in order to reject animals that present in poor condition.

### Housing and Diet

The study was performed in animal rooms provided with filtered air at a temperature of 70° F.+/−5° F. and 50% +/−20% relative humidity. Animal rooms were set to maintain a minimum of 12 to 15 air changes per hour. The room was on an automatic timer for a light/dark cycle of 12 hours on and 12 hours off with no twilight. Bed-O-Cobs® bedding was used. Bedding was changed a minimum of once per week. Cages, tops, bottles, etc. were washed with a commercial detergent and allowed to air dry. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity was recorded during the study, and the records retained.

Animals were fed with a Purina Labdiet® 5053 rodent diet, and water was provided ad libitum.

### Animal Randomization and Allocations.

Animals were randomly and prospectively divided into three (3) treatment groups prior to irradiation. Each animal was identified by an ear punch corresponding to an individual number. For more consistent identification, ear punch numbering was used rather than tagging, since tags may become dislodged during the course of the study. A cage card was used to identify each cage or label marked with the study number, treatment group number and animal numbers.

### Weights and Survival

All animals were weighed daily and their survival recorded, in order to assess possible differences in animal weight among treatment groups as an indication for mucositis severity and/or possible toxicity resulting from the treatments.

### CASAD Composition

A dioctahedral smectite, calcium aluminosilicate clay with an average particle size of less than about 80 microns was mixed with deionized water to form a suspension of 10% w/v CASAD in water.

### Mucositis Induction

Twenty-four male Syrian Golden Hamsters were randomly assigned to three equally-sized groups. On study day 0, mucositis was induced using a standardized acute radiation protocol. A single dose of radiation (40 Gy/dose) was administered to all animals on day 0. Radiation was generated with a 160 kilovolt potential (15-ma) source at a focal distance of 40 cm, hardened with a 0.35 mm Al filtration system. Irradiation targeted the left buccal pouch mucosa at a rate of 2.5 Gy/minute. Prior to irradiation, animals were anesthetized with an intraperitoneal injection of ketamine (160 mg/mL) and xylazine (8 mg/mL).

A composition comprising CASAD was given topically to the left cheek pouch three times daily either from Day 0 to Day 28 or from Day 7 to 28. The weight and general health of the animals were evaluated daily. Beginning on day 6 and continuing on alternate days for the duration of the study, oral mucositis was scored using a standard six point scale. The number of days of ulcerative mucositis was evaluated using a chi-squared test of mucositis scores of 3 or higher, and the individual daily group scores were assessed with a rank sum test. Mucositis in the left cheek pouch was evaluated clinically starting on Day 6, and continuing on alternate days until Day 28.

On Day 28, all animals were euthanized by CO₂ inhalation and death was confirmed by monitoring heartbeat in accordance with USDA guidelines.

TABLE 1-1
Study Design

| | Number | | | Dosing |
|---|---|---|---|---|
| | of | | Treatment | Volume |
| Group | Animals | Treatment | Schedule | (mL) |
| 1 | 8 males | Vehicle Control | TID; Day 0 to 28 | 0.2 mL/Dose |
| | | (WFI) | | |
| 2 | 8 males | CASAD-10% w/v | TID; Day 0 to 28* | 0.2 mL/Dose |
| 3 | 8 males | CASAD-10% w/v | TID; Day 7 to 28 | 0.2 mL/Dose |

*The first dose of CASAD on Day 0 was administered 1-2 hours following radiation.

### Mucositis Evaluation

The grade of mucositis was scored, beginning on day 6, and for every second day thereafter, through and including day 28. The effect on mucositis of each drug treatment compared to placebo was assessed. On each evaluation day, the number of animals with a blinded mucositis score of ≧3 in each drug treatment group was compared to the control group. Differences were compared on a cumulative basis and statistical significance was determined by chi-square analysis. Efficacy, in this analysis, is defined by a significant reduction in the number of days that a group of animals had ulcerations (scores 3) when compared to the control group.

### Rank Sum Differences in Daily Mucositis Scores.

For each evaluation day the scores of the control group were compared to those of the treated groups using non-parametric rank sum analysis. Treatment success was considered as a statistically significant lowering of scores in the treated group on 2 or more days from day 6 to day 28.

### Example 2

### Evaluation of Mucositis in Animals Treated with CASAD Composition

Using the twenty-four hamsters in study groups 1, 2, and 3, an oral mucositis score, weight change and survival were measured throughout the study period described above in Example 1. For the evaluation of oral mucositis, the animals were anesthetized with an inhalation anesthetic, and the left pouch everted. Oral mucositis was scored visually by comparison to a validated photographic scale, ranging from 0 for normal, to 5 for severe ulceration (clinical scoring). In descriptive terms, this mucositis scoring scale is defined in Table 2-1, below.

TABLE 2-1
Scoring of Oral Mucositis

| Score: | Description: |
|---|---|
| 0 | Pouch completely healthy. No erythema or vasodilation. |
| 1 | Light to severe erythema and vasodilation. No erosion of |
| | mucosa. |
| 2 | Severe erythema and vasodilation. Erosion of superficial |
| | aspects of mucosa leaving denuded areas. Decreased |
| | stippling of mucosa. |
| 3 | Formation of off-white ulcers in one or more places. |
| | Ulcers may have a yellow/gray due to pseudomembrane. |
| | Cumulative size of ulcers should equal about ¼ of the |
| | pouch. Severe erythema and vasodilation. |
| 4 | Cumulative seize of ulcers should equal about ½ of the |
| | pouch. Loss of pliability. Severe erythema and vasodilation. |
| 5 | Virtually all of pouch is ulcerated. Loss of pliability |
| | (pouch can only partially be extracted from mouth). |

A score of 1-2 is considered to represent a mild stage of the disease, whereas a score of 3-5 is considered to indicate moderate to severe oral mucositis. Following visual scoring, a digital image was taken of each animal's oral mucosa using a standardized technique. At the conclusion of the experiment, images were randomly numbered and scored by two independent trained observers graded the photographs in blinded fashion using the above-described scale (blinded scoring).

To examine the levels of clinically significant oral mucositis (“ulcerative oral mucositis”), as defined by presentation with open ulcers (score >3), the percentage of the number of animals presenting with an ulcer on each day of the study was determined. The percentage of animals in each group with an Oral Mucositis Score of 3 or higher on Days 6 to 28 is reported is shown in Table 2-2.

TABLE 2-2
Percent Ulceration by Day

| | Treatment Day | | | | | | | | | | | |
|---|---|---|---|---|---|---|---|---|---|---|---|---|
| | | | Day | Day | Day | | | | | | | |
| | Day 6 | Day 8 | 10 | 12 | 14 | Day 16 | Day 18 | Day 20 | Day 22 | Day 24 | Day 26 | Day 28 |
| 1 | 0.0% | 0.0% | 0.0% | 0.0% | 62.5% | 100% | 100% | 100% | 75.0% | 62.5% | 37.5% | 37.5% |
| (Control) | | | | | | | | | | | | |
| 2 | 0.0% | 0.0% | 0.0% | 0.0% | 25.0% | 87.5% | 75.0% | 75.0% | 75.0% | 62.5% | 75.0% | 87.5% |
| (10% CASAD | | | | | | | | | | | | |
| on days 0-28) | | | | | | | | | | | | |
| 3 | 0.0% | 0.0% | 0.0% | 12.5% | 62.5% | 87.5% | 87.5% | 87.5% | 87.5% | 87.5% | 87.5% | 50.0% |
| (10% CASAD | | | | | | | | | | | | |
| on days 7-28) | | | | | | | | | | | | |

All eight (100%) of the vehicle control animals developed ulcerative oral mucositis by Day 16 and this ulceration persisted through 20 days after radiation. Meanwhile, in the groups treated with CASAD there was never a study day where all animals from either treatment group exhibited ulceration. The largest treatment effects were observed in the group treated with CASAD from Day 0 to 28. Here, the percent ulceration was reduced compared to vehicle control by 37.5% on Day 14, by 12.5% on Day 16, and by 25% on Days 18 and 20.

No deaths were observed during this study. The results of daily weight change are shown in FIGS. 1A-1B. The duration of mucositis is shown in Table 1 and FIGS. 2A-2B. The severity of mucositis is reported in Table 2.

### Example 3

### Binding of the Pro-Inflammatory Cytokine with a CASAD Composition

A dioctahedral smectite, calcium aluminosilicate clay with an average particle size of less than about 80 microns was suspended in phosphate buffered saline at six different concentrations: 0.5 mg/mL, 1 mg/mL, 5 mg/mL, 10 mg/mL, 50 mg/mL, and 100 mg/mL.

Recombinant TNFα (50 mg/ml stock in 100% ddH₂O) was added to each of the CASAD samples to a final concentration of 1000 pg/mL TNFα. One sample of 1000 pg/mL TNFα in 100% PBS with no CASAD was prepared as a control. The samples were vortexed for 30 seconds and allowed to incubate at room temperature for 30 minutes. During this incubation the samples were vortexed again every 10 minutes for 5 seconds. After incubation, the samples were centrifuged at 10,000 rpm for 5 minutes and the supernatant was isolated.

Follow the protocol on a TNFα ELISA kit (R&D Systems Inc.), the amount of TNF-alpha in each supernatant was examined. The results are shown in FIGS. 3A-3B.

While a number of exemplary aspects and embodiments have been discussed and illustrated, those of skill in the art will recognize certain modifications, permutations, additions and sub-combinations thereof which do not depart from the scope of the present disclosure. It is to be understood that the disclosure is not to be limited to the specific embodiments disclosed herein, as such are presented by way of example. It is therefore intended that the following appended claims and claims hereafter introduced are interpreted to include all such modifications, permutations, additions and sub-combinations as are within their true spirit and scope.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

It is claimed:

1. A method of treating oral mucositis, comprising:
topically administering to epithelial tissue within the oral cavity of a subject in need thereof a therapeutically effective amount of a composition comprising calcium aluminosilicate clay with an average particle size of less than about 100 microns (CASAD).

2. The method of claim 1, wherein the CASAD has a selected average particle size that is achieved by sieving or by air classification of the CASAD.

3. The method of claim 2, wherein the CASAD is non-swelling and a calcium species as evidenced by a shrink/swell potential (SV) of less than 1.5 COLE index value.

4. The method of claim 1, wherein the administering comprises administering to a subject with cancer.

5. The method of claim 1, wherein the administering comprises administering to a subject undergoing or planning to undergo chemotherapy.

6. The method of claim 1, wherein the administering is prior to or concurrent with initiation of radiation therapy in the cancer subject.

7. The method of claim 1, wherein the administering is after radiation therapy.

8. The method of claim 1, wherein the administering continues for the duration of radiation therapy.

9. The method of claim 1, wherein the administering comprises administering more than once daily.

10. The method of claim 1, wherein the administering comprises administering once daily.

11. The method of claim 1, wherein the administering comprises administering orally a fluid comprising the CASAD.

12. The method of claim 11, wherein the fluid is a solution, a suspension, a paste, or a gel.

13. The method of claim 11, wherein administering comprises administering the fluid wherein the fluid is held in the mouth for a recommended period of time.

14. The method of claim 11, wherein the method further comprises discharging the fluid from the mouth.

15. The method of claim 1, wherein the administering comprises administering a composition comprising a polymer.

16. The method of claim 1, wherein the administering comprises administering a solid dosage form that disintegrates in an aqueous medium.

17. The method of claim 1, wherein the subject is concurrently treated with at least one therapeutic agent.

18. The method of claim 17, wherein the therapeutic agent is selected from the group consisting of a pain reliever, a chemotherapeutic, an anti-inflammatory and an antibiotic.

19. A method to reduce or delay the onset of oral mucositis, comprising:
topically administering to epithelial tissue within the oral cavity of a subject in need thereof a therapeutically effective amount of a composition comprising calcium aluminosilicate clay with an average particle size of less than about 100 microns (CASAD).

20. A method for preventing oral mucositis in a subject, comprising:
topically administering to epithelial tissue within the oral cavity of a subject in need thereof a therapeutically effective amount of a composition comprising calcium aluminosilicate clay with an average particle size of less than about 100 microns (CASAD).

21. A method for reducing severity of oral mucositis in a subject, comprising:
topically administering to epithelial tissue within the oral cavity of a subject in need thereof a therapeutically effective amount of a composition comprising calcium aluminosilicate clay with an average particle size of less than about 100 microns (CASAD).
